(19) United States
(12) Patent Application Publication
(10) Pub. No.: US 2022/0160615 A1
(43) Pub. Date: May 26, 2022

(54) COSMETIC, COSMETIC FOR USE IN WARMED STATE, AND BEAUTY METHOD

(71) Applicant: Shiseido Company, Ltd., Tokyo (JP)

(72) Inventor: Takahiro SUZUKI, Tokyo (JP)

(73) Assignee: Shiseido Company, Ltd., Tokyo (JP)

(21) Appl. No.: 17/434,829

(22) PCT Filed: Feb. 26, 2020
(86) PCT No.: PCT/JP2020/007639

§ 371 Date: Aug. 30, 2021

(30) Foreign Application Priority Data
Mar. 4, 2019 (JP) 2019-038193

(51) Int. Cl. A61K 8/87; A61K 8/73; A61K 8/02; A61Q 19/00; A45D 34/04

(52) U.S. Cl. CPC A61K 8/87; A61K 8/731; A61K 8/027; A61Q 19/00; A45D 2200/155; A61K 2800/87; A61K 2800/54; A61K 2800/48; A61K 2800/10; A45D 34/04

(57) ABSTRACT

To provide a cosmetic that suppresses greasiness and siphons continuously during a dispenser operation. Also, to provide a cosmetic for use in a warmed state, the cosmetic having temperature responsiveness and high high-temperature stability. The cosmetic contains (A) a hydrophobically modified polyether urethane, (B) cellulose nanofibers, and (C) water, wherein: when the blend ratio of (A) and (B) is such that (A)<(B), the amount of (A)+(B) blended is 0.75% by mass or less relative to the total cosmetic; when the blend ratio of (A) and (B) is such that (A)=(B), the amount of (A)+(B) blended is 1.75% by mass or less relative to the total cosmetic; and when the blend ratio of (A) and (B) is such that (A)>(B), the amount of (A)+(B) blended is 2% by mass or less relative to the total cosmetic.

### CROSS-REFERENCE TO RELATED APPLICATIONS

This application is a National Phase Entry of PCT International Application No. PCT/JP2020/007639 filed on Feb. 26, 2020, which claims priority under 35 U.S.C. § 119(a) to Japanese Patent Application No. 2019-038193 filed on Mar. 4, 2019.

### BACKGROUND

The present disclosure relates to a cosmetic comprising a hydrophobic polyether urethane and cellulose nanofibers, a cosmetic for use in a warmed state, comprising a temperature-responsive polymer and a high-temperature-stable polymer, and a beauty method.

In order to improve the stability of a preparation and the pleasantness of usability, water-soluble thickeners have conventionally been added to cosmetics, and the types, blend amount, and combination of the thickeners are adjusted in accordance with the applications intended by users. For example, Japanese Unexamined Patent Publication No. 2017-048181 describes cosmetics comprising cellulose nanocrystals and water-soluble polymers such as carboxy vinyl polymers, alkyl methacrylate/acrylic acid copolymers, and thickening polysaccharides.

In general, when a thickener is added to a cosmetic, the concentration of the thickener rises due to the volatilization of the solvent contained in the cosmetic. For this reason, remarkable greasiness attributable to the thickener is caused, resulting in deteriorated usability. When the amount of a thickener blended to a cosmetic is increased, the thixotropic properties of the cosmetic increase in general. As a result, there is a decrease in the usable amount of the cosmetic due to the increased amount of the cosmetic adhering onto a container wall, and poor siphoning by a dispenser occurs. Particularly when being siphoned with a dispenser, there is a problem such that cosmetics are not continuously siphoned, resulting in discontinuous dispensing with mixed air.

Cosmetics are contained in various forms of containers such as bottles, tubes, jars, and mist dispensers. In the development of cosmetics regulated with water-soluble thickeners, not only the selection of thickeners but also the design of cosmetics by considering the shapes of containers is important.

Conventionally, it has been proven that higher actual feelings of effectiveness in use can be realized when a cosmetic is used in a warmed state, compared with the use of a cosmetic at room temperature, and the development of cosmetics used in a warmed state (hereinafter also referred to as “cosmetics for use in a warmed state”) has been started. For the cosmetics for use in a warmed state, a temperature-responsive base, having its afterfeel controlled by utilizing its physical properties which change in accordance with temperatures, is suitable.

However, the use of a simply warmed conventional cosmetic sometimes causes problems in stability such as dripping of the cosmetic during use due to its reduced viscosity, and separation thereof. In order to satisfy use conditions where users are satisfied with a cosmetic, the components to be blended, particularly the thickeners for adjusting the viscosity of the cosmetic, must be selected.

As cosmetics for use in a warmed state, those adjusted with water-soluble thickeners have been developed. For example, Japanese Unexamined Patent Publication No. 2012-240926 describes a cosmetic having its afterfeel adjusted by using a combination of a temperature-responsive polymer and a water-soluble thickener.

### SUMMARY

The disclosure of Japanese Unexamined Patent Publication No. 2017-048181 adopts a specific water-soluble polymer in order to achieve a homogeneous cosmetic by suppressing aggregation of fine cellulose. However, by considering aspects relevant to containers such as increased amount of a cosmetic adhering to a container wall and poor siphoning with a dispenser, thickeners are not selected.

Meanwhile, in the field of cosmetics for use in a warmed state, using temperature-responsive water-soluble thickeners and increasing high temperature stability without reducing the temperature responsiveness have not been considered.

In view of the foregoing circumstances, the present disclosure is directed first to provide a cosmetic of which greasiness has been eliminated by having film-forming properties when being dried, and also which is continuously siphoned when a dispenser is operated.

Secondly, the present disclosure is directed to provide a cosmetic for use in a warmed state which has high high-temperature stability while maintaining temperature responsiveness. The present disclosure is further directed to provide a beauty method by applying cosmetics for use in a warmed state.

The cosmetic according to the present disclosure is

a cosmetic comprising

(A) a hydrophobically modified polyether urethane,

(B) cellulose nanofibers, and

(C) water,

in which,

when the blend ratio of the (A) hydrophobically modified polyether urethane to the (B) cellulose nanofibers is such that (A)<(B), the amount of (A)+(B) blended relative to the total cosmetic is 0.75% by mass or lower,

when the blend ratio of (A) to (B) is such that (A)=(B), the amount of (A)+(B) blended relative to the total cosmetic is 1.75% by mass or lower, and

when the blend ratio of (A) to (B) is such that (A)>(B), the amount of (A)+(B) blended relative to the total cosmetic is 2% by mass or lower.

The (A) hydrophobically modified polyether urethane is preferably a (PEG-240/decyltetradeces-20/HDI) copolymer. PEG is an abbreviation for polyethylene glycol, and HDI is an abbreviation for hexamethylene diisocyanate.

The (B) cellulose nanofibers are preferably microfibrous cellulose having a maximum fiber diameter of 1,000 nm or less.

The cosmetic according to the present disclosure is preferably contained in a dispenser container.

The cosmetic for use in a warmed state according to the present disclosure is

a cosmetic for use in a warmed state that is used in a device having a heating part, said cosmetic comprising

a temperature-responsive polymer having a structure changing at a temperature of 30° C. or higher,

a high-temperature-stable polymer having a structure not changing at a temperature of 70° C. or lower, and

water.

The temperature-responsive polymer is preferably (A) a hydrophobically modified polyether urethane and the high-temperature-stable polymer is preferably (B) cellulose nanofibers.

The amount of the temperature-responsive polymer blended is preferably higher than the amount of the high-temperature-stable polymer blended, and the amount of the high-temperature-stable polymer blended relative to the total cosmetic is preferably 0.1% by mass or higher.

The (A) hydrophobically modified polyether urethane is preferably a (PEG-240/decyltetradeces-/HDI) copolymer.

The (B) cellulose nanofibers preferably consist of microfibrous cellulose having a maximum fiber diameter of 1,000 nm or less.

The cosmetic for use in a warmed state according to the present disclosure is preferably used under temperature conditions at 30 to 70° C.

The heating part preferably has a heat source which is a heater or a Peltier device.

The device may be equipped with an atomizer.

The device may be equipped with a probe.

The device may be equipped with a tank for containing the cosmetic for use in a warmed state.

The beauty method according to the present disclosure is a method in which the temperature of the above cosmetic for use in a warmed state is controlled within a temperature range of 40 to 70° C. in the heating part and the cosmetic is directly and/or indirectly applied in a mist state onto the skin.

The beauty method according to the present disclosure is a method in which the temperature of the above cosmetic for use in a warmed state is controlled within a temperature range of 30 to 48° C. in the heating part and the cosmetic is directly and/or indirectly applied onto the skin.

Due to the cosmetic according to the present disclosure, comprising

(A) a hydrophobically modified polyether urethane,

(B) cellulose nanofibers, and

(C) water,

in which,

when the blend ratio of the (A) hydrophobically modified polyether urethane to the (B) cellulose nanofibers is such that (A)<(B), the amount of (A)+(B) blended relative to the total cosmetic is 0.75% by mass or lower,

when the blend ratio of (A) to (B) is such that (A)=(B), the amount of (A)+(B) blended relative to the total cosmetic is 1.75% by mass or lower, and

when the blend ratio of (A) to (B) is such that (A)>(B), the amount of (A)+(B) blended relative to the total cosmetic is 2% by mass or lower,

the cosmetic has film-forming properties, and thereby greasiness is eliminated, and is also continuously siphoned when a dispenser is operated.

Since the cosmetic for use in a warmed state according to the present disclosure is a cosmetic for use in a warmed state which is used in a device having a heating part, and comprises

a temperature-responsive polymer having a structure changing at a temperature of 30° C. or higher,

a high-temperature-stable polymer having a structure not changing at a temperature of 70° C. or lower, and

water,

the cosmetic can have high high-temperature stability while maintaining temperature responsiveness.

### BRIEF DESCRIPTION OF THE DRAWINGS

FIG. 1 A graph showing a relationship between the elastic modulus and distortion of a cosmetic for use in a warmed state when the ratio of (A) to (B) is 100 to 0 ((A):(B)=100:0).

FIG. 2 A graph showing a relationship between the elastic modulus and distortion of a cosmetic for use in a warmed state when the ratio of (A) to (B) is 75 to 25 ((A):(B)=75:25).

FIG. 3 A graph showing a relationship between the elastic modulus and distortion of a cosmetic for use in a warmed state when the ratio of (A) to (B) is 50 to 50 ((A):(B)=50:50).

FIG. 4 A graph showing a relationship between the elastic modulus and distortion of a cosmetic for use in a warmed state when the ratio of (A) to (B) is 25 to 75 ((A):(B)=25:75).

FIG. 5 A graph showing a relationship between the elastic modulus and distortion of a cosmetic for use in a warmed state when the ratio of (A) to (B) is 0 to 100 ((A):(B)=0:100).

### DETAILED DESCRIPTION OF THE PREFERRED EMBODIMENTS

First, cosmetics according to the present disclosure are described. A cosmetic according to the present disclosure is one comprising

(A) a hydrophobically modified polyether urethane (hereinafter simply referred to as “(A)”),

(B) cellulose nanofibers (hereinafter simply referred to as “(B)”), and

(C) water,

in which,

when the blend ratio of (A) to (B) is such that (A)<(B), the amount of (A)+(B) blended relative to the total cosmetic is 0.75% by mass or lower,

when the blend ratio of (A) to (B) is such that (A)=(B), the amount of (A)+(B) blended relative to the total cosmetic is 1.75% by mass or lower, and

when the blend ratio of (A) to (B) is such that (A)>(B), the amount of (A)+(B) blended relative to the total cosmetic is 2% by mass or lower.

Each component is described below.

(A) Hydrophobically Modified Polyether Urethane

(A) A hydrophobically modified polyether urethane is represented by the formula (I) below. The copolymer is an associative thickener which is known to have temperature responsiveness. An associative thickener is a copolymer having a hydrophilic group moiety as a backbone and a hydrophobic moiety at an end, and is such that the hydrophilic moieties of the copolymer associate with each other in an aqueous medium to exhibit a thickening effect. The thickening effect of the associative thickeners as such is exhibited by the mechanism such that the hydrophobic moieties of a copolymer associate with each other in an aqueous medium and hydrophilic moieties form a loop and a bridge.

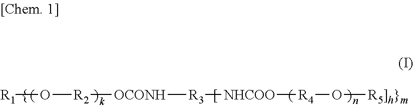

In the above formula (I), R₁, R₂, and R₄ are respectively and independently an alkylene group having 2 to 4 carbon atoms or a phenylethylene group, preferably an alkylene group having 2 to 4 carbon atoms.

R₃ is an alkylene group having 1 to 10 carbon atoms, which may have a urethane bond.

R₅ is a linear, branched, or secondary alkyl group having 8 to 36, preferably 12 to 24 carbon atoms.

m is a number of 2 or more, preferably 2.

h is a number of 1 or more, preferably 1.

k is a number of 1 to 500, preferably 100 to 300.

n is a number of 1 to 200, preferably 10 to 100.

A preferred example of a method for obtaining a hydrophobically modified polyether urethane represented by the above formula (I) is such that a reaction among one or more polyether polyols represented by R₁—[(O—R₂)ₖ—OH]ₘ (in which R₁, R₂, k, and m are as defined above), one or more polyisocyanates represented by R₃—(NCO)ₕ₊₁ (in which R₃ and h are as defined above), and one or more polyether monoalcohols represented by HO—(R₄—O)ₙ—R₅ (in which R₄, R₅, and n are as defined above) is effectuated.

In the above example, R₁ to R₅ in the formula (I) are determined in accordance with the R₁—[(O—R₂)ₖ—OH]ₘ, R₃—(NCO)ₕ₊₁, and HO—(R₄—O)ₙ—R₅ used. The feed ratio of the above three components are not particularly limited and is preferably such that isocyanate groups derived from polyisocyanates to the ratio of hydroxy groups derived from polyether polyols and polyether monoalcohols is 0.8:1 to 1.4:1 (NCO/OH=0.8:1 to 1.4:1).

Polyether polyol compounds represented by the above formula R₁—[(O—R₂)ₖ—OH]ₘ are prepared by addition polymerization of an alkylene oxide such as ethylene oxide, propylene oxide, butylene oxide, or epichlorohydrin, or a styrene oxide to an m-valent polyol.

Here, 2- to 8-valent polyols are preferred. Examples thereof are bivalent alcohols such as ethylene glycol, propylene glycol, butylene glycol, hexamethylene glycol, and neopentyl glycol; trivalent alcohols such as glycerin, trioxyisobutane, 1,2,3-butanetriol, 1,2,3-pentatriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4, 5-heptanetriol, 2,4-dimethyl-2,3,4-pentanetriol, pentamethyl glycerin, pentaglycerin, 1,2,4-butanetriol, 1,2,4-pentanetriol, trimethylolethane, and trimethylolpropane; tetravalent alcohols such as pentaerythritol, 1,2,3,4-pentanetetrol, 2,3,4,5-hexanetetrol, 1,2,4,5-pentanetetrol, and 1,3,4,5-hexanetetrol; pentavalent alcohols such as adonitol, arabitol, and xylitol; hexavalent alcohols such as dipentaerythritol, sorbitol, mannitol, and iditol; and octavalent alcohols such as sucrose.

R₂ is determined in accordance with an alkylene oxide or a styrene oxide to be addition polymerized. Particularly, alkylene oxides having 2 to 4 carbon atoms or styrene oxide is preferred since these are easily available and allow excellent effects to be exhibited.

Alkylene oxides or styrene oxide may be added through homopolymerization, random polymerization of 2 of more thereof, or block polymerization. The addition polymerization may be performed by an ordinary method. The degree of polymerization k is 1 to 500. The ratio of ethylene groups in R₂ is preferably 50 to 100% by mass relative to the total of R₂.

The molecular weight of R₁—[(O—R₂)ₖ—OH]ₘ is preferably 500 to 100,000, and is particularly preferably 1,000 to 50,000.

Polyisocyanates represented by the above formula R₃—(NCO)ₕ₊₁ are not particularly limited as long as they have 2 or more isocyanate groups in a molecule. Examples thereof are aliphatic diisocyanates, aromatic diisocyanates, alicyclic diisocyanates, biphenyl diisocyanates, di-, tri-, and tetra isocyanates of phenyl methane.

Examples of aliphatic diisocyanates are methylene diisocyanate, dimethylene diisocyanate, trimethylene diisocyanate, tetramethylene diisocyanate, pentamethylene diisocyanate, hexamethylene diisocyanate, dipropylether diisocyanate, 2,2-dimethylpentane diisocyanate, 3-methoxyhexane diisocyanate, octamethylene diisocyanate, 2,2,4-trimethylpentane diisocyanate, nonamethylene diisocyanate, decamethylene diisocyanate, 3-butoxyhexane diisocyanate, 1,4-butyleneglycol dipropyl ether diisocyanate, thiodihexyl diisocyanate, meta-xylylene diisocyanate, para-xylylene diisocyanate, and tetramethylxylylene diisocyanate.

Examples of aromatic diisocyanates are meta-phenylene diisocyanate, para-phenylene diisocyanate, 2,4-tolylene diisocyanate, 2,6-tolylene diisocyanate, dimethylbenzene diisocyanate, ethylbenzene diisocyanate, isopropylbenzene diisocyanate, tolidine diisocyanate, 1,4-naphthalene diisocyanate, 1,5-naphthalene diisocyanate, 2,6-naphthalene diisocyanate, and 2,7-naphthalene diisocyanate.

Examples of alicyclic diisocyanates are hydrogenated xylylene diisocyanate and isophorone diisocyanate.

Examples of biphenyl diisocyanates are biphenyl diisocyanate, 3,3′-dimethylbiphenyl diisocyanate, and 3,3′-dimethoxybiphenyl diisocyanate.

Examples of diisocyanates of phenyl methane are diphenylmethane-4,4′-diisocyanate, 2,2′-dimethyldiphenylmethane-4,4′-diisocyanate, diphenyldimethylmethane-4,4′-diisocyanate, 2,5,2′,5′-tetramethyldiphenylmethane-4,4′-diisocyanate, cyclohexylbis(4-isosiontophenyl)methane, 3,3′-dimethoxydiphenylmethane-4,4′-diisocyanate, 4,4′-dimethoxydiphenylmethane-3,3′-diisocyanate, 4,4′-diethoxydiphenylmethane-3,3′-diisocyanate, 2,2′-dimethyl-5,5′-dimethoxydiphenylmethane-4,4′-diisocyanate, 3,3′-dichlorodiphenyldimethylmethane-4,4′-diisocyanate, and benzophenone-3,3′-diisocyanate.

Examples of triisocyanates of phenylmethane are 1-methylbenzene-2,4,6-triisocyanate, 1,3,5-trimethylbenzene-2,4,6-triisocyanate, 1,3,7-naphthalenetriisocyanate, biphenyl-2,4,4′-triisocyanate, diphenylmethane-2,4,4′-triisocyanate, 3-methyldiphenylmethane-4,6,4′-triisocyanate, triphenylmethane-4,4′,4″-trisocyanate, 1,6,11-undecanetriisocyanate, 1,8-diisocyanate-4-isocyanatemethyloctane, 1,3,6-hexamethylene triisocyanate, bicycloheptane triisocyanate, and tris(isocyanatephenyl)thiophosphate.

The above polyisocyanate compounds may be used in a form of a dimer, or a trimer (isocyanurate bond), and also in a form of biuret by reacting with an amine.

In addition, polyisocyanates having a urethane bond obtained by reacting the above polyisocyanate compounds with polyols may also be used. As polyols, 2 to 8 valent polyols are preferred and the above polyols are preferred. When a trivalent or more polyisocyanate is used as R₃—(NCO)ₕ₊₁, the polyisocyanate having a urethane bond is preferred.

Polyether monoalcohols represented by the above formula HO—(R₄—O)ₙ—R₅ are not particularly limited as long as they are polyethers of linear, branched, or secondary monovalent alcohols. The compounds as such are obtainable by addition polymerization of an alkylene oxide such as ethylene oxide, propylene oxide, butylene oxide, or epichlorohydrin, or styrene oxide to a linear, branched, or secondary monovalent alcohol.

Linear alcohols are herein represented by the following formula (II).

R₆—OH (II)

Branched alcohols are herein represented by the following formula (III).

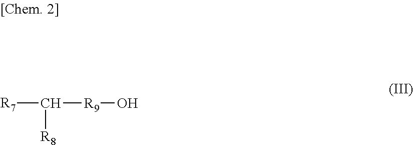

In addition, secondary alcohols are represented by the following formula (IV).

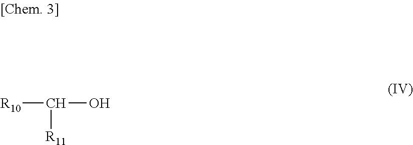

Thus, R₅ is a group represented by the above formula (II), (III), or (IV) from which a hydroxy group is removed. In the above formulae (II) to (IV), R₆, R₇, R₈, R₁₀, and R₁₁ are hydrocarbon groups or fluorocarbon groups such as alkyl groups, alkenyl groups, alkyl aryl groups, cycloalkyl groups, and cycloalkenyl groups.

Examples of alkyl groups are methyl, ethyl, propyl, isopropyl, butyl, isobutyl, tertiary butyl, pentyl, isopentyl, neopentyl, tertiary pentyl, hexyl, heptyl, octyl, 2-ethylhexyl, nonyl, decyl, undecyl, dodecyl, tridecyl, isotridecyl, myristyl, palmityl, stearyl, isostearyl, icosyl, docosyl, tetracosyl, triacontyl, 2-octyldodecyl, 2-dodecylhexadecyl, 2-tetradecyloctadecyl, and monomethyl branched-isostearyl.

Examples of alkenyl groups are vinyl, allyl, propenyl, isopropenyl, butenyl, pentenyl, isopentenyl, hexenyl, heptenyl, octenyl, nonenyl, decenyl, undecenyl, dodecenyl, tetradecenyl, and oleyl.

Examples of alkyl aryl groups are phenyl, tolyl, xylyl, cumenyl, mesityl, benzyl, phenethyl, styryl, cinnamyl, benzhydryl, trityl, ethylphenyl, propylphenyl, butylphenyl, penthylphenyl, hexylphenyl, heptylphenyl, octylphenyl, nonylphenyl, α-naphthyl, and β-naphthyl.

Examples of cycloalkyl groups and cycloalkenyl groups are cyclopentyl, cyclohexyl, cycloheptyl, methylcyclopentyl, methylcyclohexyl, methylcycloheptyl, cyclopentenyl, cyclohexenyl, cycloheptenyl, methylcycloheptenyl, methylcyclohexenyl, and methylcycloheptenyl.

In the above formula (III), R₉ is hydrocarbon or fluorocarbon, and examples thereof are alkylene, alkenylene, alkyl arylene, cyclalkylene, and cycloalkenylene.

R₅ is hydrocarbon or fluorocarbon, and preference is given to alkyl, and further to alkyl having 8 to 36 carbon atoms, and particularly preferably 12 to 24 carbon atoms in total.

Alkylene oxides or styrene oxide may be added through homopolymerization, random polymerization of 2 or more thereof, or block polymerization. The addition polymerization may be performed by an ordinary method. The degree of polymerization n is 0 to 1,000, preferably 1 to 200, and more preferably 10 to 200. The ratio of ethylene groups in R₄ is preferably 50 to 100% by mass, and more preferably 65 to 100% by mass relative to the total of R₄, in order to obtain a good associative thickener suitable for the purpose of the present disclosure.

Copolymers represented by the above formula (I) may be prepared by a method similar to an ordinary reaction between a polyether and an isocyanate, for example, by performing heating at a temperature of 80 to 90° C. for 1 to 3 hours to effectuate a reaction.

When a polyether polyol (a) represented by R₁—[(O—R₂)ₖ—OH]ₘ, a polyisocyanate (b) represented by R₃—(NCO)ₕ₊₁, and a polyether monoalcohol (c) represented by HO—(R₄—O)ₙ—R₅ are reacted, by-products other than copolymers having the structure of the formula (I) may be produced. For example, when a diisocyanate is used, a c-b-a-b-c type copolymer represented by the formula (I) is produced as a main product, and other copolymers of types such as a c-b-c type, and a c-b-(a-b)x-a-b-c type may be produced. In this case, the obtained mixture containing the copolymer of the formula (I) may be used in the present disclosure without particularly separating the copolymer of the formula (I).

A particularly preferred example is a hydrophobically modified polyether urethane having the INCI name “(PEG-240)/decyltetradeceth-20/HDI) copolymer (PEG-240/HDI COPOLYMER BISDECYLTETRADECETH-20 ETHER)”. Said copolymer is commercially available as “ADEKANOL GT-700” (trade name) from ADEKA CORPORATION.

(B) Cellulose Nanofibers

Cellulose nanofiber means a fiber obtained by defibrating a cellulose fiber derived from a plant cell wall to the nano-level, and a fine fibrous cellulose having a maximum fiber diameter of 1,000 nm or less is preferred. Further in detail, preference is given to a fine cellulose fiber having a number average fiber diameter of 2 to 100 nm, in which the cellulose has a cellulose type I crystal structure, a hydroxy group in C6 position of a glucose unit in a cellulose molecule is selectively oxidized and modified into an aldehyde group or a carboxyl group, and the amount of carboxyl groups is 0.6 to 2.2 mmol/g. This means that the above cellulose fiber is a fiber in which a native cellulose solid raw material having a type I crystal structure is surface oxidized and micronized. In other words, during the biosynthesis of native cellulose, nanofibers called microfibrils are first formed nearly with no exception, and the thus-formed microfibrils form multiple bundles to constitute a higher order solid structure, in which part of hydroxy groups are oxidized in order to weaken the hydrogen bond between surfaces which causes a strong cohesion between the microfibrils, and are converted into an aldehyde group and a carboxy group.

Here, the type I crystal structure of a cellulose constituting the cellulose nanofibers is identified based on a diffraction profile obtained by a wide angle X ray diffraction in which typical peaks are observed in two positions near 2θ=14 to 17° and 2θ=22 to 23°.

The cellulose nanofibers have a maximum fiber diameter of 1,000 nm or less and a number average fiber diameter of 2 to 100 nm, and preferably have a number average fiber diameter of 3 to 80 nm from the viewpoint of dispersion stability. Namely, when the above number average fiber diameter is 2 nm or more, the dissolution in a dispersion medium is more suppressible, and when the number average fiber diameter is 100 nm or less, and similarly with a maximum fiber diameter of 1,000 nm or less, cellulose fiber precipitation is suppressed and functionality achieved by mixing cellulose fibers can be sufficiently expressed.

The number average fiber diameter and the maximum fiber diameter of the cellulose nanofibers are measurable in the following manner, for example. Water is added to cellulose fibers until the solids in the cellulose are 1% by mass. The obtained solution was dispersed with an ultrasonic homogenizer, a high-pressure homogenizer, or a blender having a rotational velocity capacity of 15,000 rpm or higher, and thereafter, the obtained dispersion was freeze-dried to prepare a sample. The thus-obtained sample was observed with a scanning electron microscope (SEM), and based on the obtained images, the number average fiber diameter and the maximum fiber diameter of the cellulose fibers can be measured and calculated.

Preference is given to cellulose nanofibers in which a hydroxy group in C6 position of a glucose unit in a cellulose molecule is selectively oxidized and is modified into an aldehyde group or a carboxyl group, and the amount of carboxyl groups is 0.6 to 2.2 mmol/g. Moreover, the amount of carboxyl groups is particularly preferably within a range of 0.6 to 2.0 mmol/g in terms of shape-retention properties and dispersion stability. Namely, when the above carboxyl group amount is 0.6 mmol/g or higher, the dispersion stability of cellulose fibers becomes better and precipitation is suppressible. When the carboxyl group amount is 2.2 mmol/g or lower, water solubility is appropriately maintained and greasy afterfeel is suppressible.

The amount of carboxyl groups in cellulose nanofibers may be measured for example by potentiometric titration. Namely, dry cellulose fibers are dispersed in water, a 0.01N aqueous sodium chloride solution is added thereto and is sufficiently stirred to disperse cellulose fibers. Next, a 0.1 N hydrochloric acid solution was added to achieve a pH value of 2.5 to 3.0, and a 0.04 N aqueous sodium hydroxide solution is added dropwise at a rate of 0.1 mL per minute. Based on the difference between the point of neutralization of excessive hydrochloric acid and the point of neutralization of the cellulose fiber-derived carboxyl groups shown on the obtained pH curve, the amount of carboxyl groups can be calculated.

The amount of carboxyl groups is adjustable by controlling the addition amount of a co-oxidizing agent used in the step of oxidizing cellulose fibers and reaction time as described later.

Preference is given to cellulose nanofibers in which only the hydroxy group in C6 position of a glucose unit on a cellulose fiber surface is selectively oxidized into an aldehyde group or a carboxyl group. Whether only the hydroxy group in C6 position of a glucose unit on a cellulose fiber surface is selectively oxidized into an aldehyde group or a carboxyl group, can be confirmed by the ¹³C-NMR chart, for example. Namely, the peak of 62 ppm corresponding to a primary hydroxy group in C6 position of a glucose unit of cellulose confirmed by the ¹³C-NMR chart before oxidization disappears after oxidization, and instead, a peak derived from carboxyl groups appears at 178 ppm. Thereby it can be confirmed that only the hydroxy group in C6 position of a glucose unit is oxidized into an aldehyde group or a carboxyl group.

Cellulose nanofibers are prepared for example as described below. First, a native cellulose such as a soft wood pulp is dispersed in water to form a slurry, sodium bromide and an N-oxyradical catalyst are added thereto, which are sufficiently stirred to be dispersed and dissolved therein. Subsequently, a co-oxidizing agent such as an aqueous hypochlorous acid solution is added while adding dropwise a 0.5 N aqueous sodium hydroxide solution so as to maintain a pH of 10.5, where the reaction is effectuated until the change in pH is no longer observed. The slurry obtained by the above reaction is purified by water washing and filtration to remove the unreacted raw materials and catalyst. Thereby a water dispersion of specific cellulose fibers having oxidized fiber surfaces is obtainable. When higher transparency is required for cosmetics, dispersing devices having strong dispersing force such as a high-pressure homogenizer and an ultra-high pressure homogenizer are used in the treatment to achieve cosmetics having better transparency.

Examples of the above N-oxyradical catalyst are 2,2,6,6-tetramethylpiperidino-oxyradical (TEMPO), and 4-acetamide-TEMPO. The N-oxyradical catalyst is adequate when it is added in a catalytic amount, and is added to the aqueous reaction solution in an amount within a range preferably of 0.1 to 4 mmol/L, and more preferably of 0.2 to 2 mmol/L.

Examples of the above co-oxidizing agent are hypohalous acids or salts thereof, halogenous acids or salts thereof, perhalogenous acids or salts thereof, hydrogen peroxides, and perorganic acids. These may be used alone or in combination of 2 or more thereof. Among them, alkali metal hypohalous acids such as sodium hypochlorite, and sodium hypobromite are preferred. When the above sodium hypochlorite is used, it is preferred in terms of reaction rate to advance a reaction in the presence of a brominated alkali metal such as sodium bromide. The brominated alkali metal is added in a molar quantity of approximately 1 to 40 times, and preferably approximately 10 to 20 times that of the N-oxyradical catalyst.

Commercially available cellulose nanofibers may also be used, and “REOCRYSTA C-25P” (trade name) available from DSK Co., Ltd., may be used for example.

When the blend ratio of the component (A) to the component (B) is such that (A)<(B), the amount of (A)+(B) blended relative to the total cosmetic is 0.75% by mass or lower; when (A)=(B), the amount of (A)+(B) blended relative to the total cosmetic is 1.75% by mass or lower; and when (A)>(B), the amount of (A)+(B) blended relative to the total cosmetic is 2% by mass or lower. Due to the amount of (A)+(B) blended relative to the total cosmetics being not higher than the above values, film-forming properties are imparted when being dried, and cosmetics which are not greasy, and continuously siphoned during the operation of a dispenser are achieved as well.

When the blend ratio of the component (A) to the component (B) is such that (A)<(B), the amount of (A)+(B) blended relative to the total cosmetic is more preferably within a range of 0.01 to 0.75% by mass, and still more preferably within a range of 0.1 to 0.5% by mass. When (A)=(B), the amount of (A)+(B) blended relative to the total cosmetic is more preferably within a range of 0.02 to 1.75% by mass, and still more preferably within a range of 0.2 to 1.5% by mass. When (A)>(B), the amount of (A)+(B) blended relative to the total cosmetic is more preferably within a range of 0.02 to 2% by mass, and still more preferably within a range of 0.2 to 1.75% by mass.

Since the cosmetic according to the present disclosure is continuously siphoned during a dispenser operation, it can be suitably used in an embodiment in which a cosmetic is contained in a dispenser container. The dispenser container means a container from which a predetermined amount of the content of the container can be removed by pressing a press button provided on the head part of the container without inclining the container.

Subsequently, cosmetics for use in a warmed state according to the present disclosure are described. A cosmetic for use in a warmed state according to the present disclosure is a cosmetic for use in a warmed state that is used in a device having a heating part, comprising

a temperature-responsive polymer having a structure changing at a temperature of 30° C. or higher,

a high-temperature-stable polymer having a structure not changing at a temperature of 70° C. or lower, and

water.

Each component is described below.

(Temperature-Responsive Polymer Having a Structure Changing at a Temperature of 30° C. or Higher)

A temperature-responsive polymer having a structure changing at a temperature of 30° C. or higher (this may hereinafter be simply referred to as temperature-responsive polymer) means that a polymeric structure swells and shrinks at a temperature of 30° C. or higher. Particularly, it means a polymer whose structure changes such that hydrophobic bonds in a molecule or between molecules in the polymer are strengthened, causing aggregation of polymeric chains. By containing a temperature-responsive polymer, viscosity reduction of a cosmetic can occur by warming. The temperature range where structural change of a temperature-responsive polymer occurs is more preferably from 30° C. or higher to lower than 80° C.

A temperature-responsive polymer is preferably (A) a hydrophobically modified polyether urethane, the details of which are the same as those described above.

(High-Temperature-Stable Polymer Having a Structure not Changing at a Temperature of 70° C. or Lower)

A high-temperature-stable polymer having a structure not changing at a temperature of 70° C. or lower (this may hereinafter be simply referred to as high-temperature-stable polymer) means that a polymeric structure does not swell and shrink at a temperature of 70° C. or lower. Particularly, it means a polymer whose structure does not change since aggregation in a molecule or between molecules of the polymer does not occur at a temperature of 70° C. or lower. By containing a high-temperature-stable polymer, viscosity reduction of a cosmetic does not occur by warming, and hence stability without dripping or separating during use can be ensured. The temperature range where structural change of a high-temperature-stable polymer does not occur is more preferably from 30° C. or higher to lower than 70° C.

A high-temperature-stable polymer is preferably (B) cellulose nanofibers, the details of which are the same as those described above.

By using a temperature-responsive polymer and a high-temperature-stable polymer simultaneously, a cosmetic for use in a warmed state which is responsive to temperature while ensuring high temperature stability before and after warming can be prepared. Particularly before warming, the physical properties of both polymers are exhibited, but after warming, the physical properties of the high-temperature-stable polymer are mainly expressed. Thereby, a cosmetic which can ensure high temperature stability while imparting a change by warming can be obtained.

It is preferred that the amount of a temperature-responsive polymer blended is higher than the amount of a high-temperature-stable polymer blended, and the amount of a high-temperature-stable polymer blended is 0.1% by mass or higher, more preferably within a range of 0.1 to 1% by mass relative to the total cosmetic. When the amount of a high-temperature-stable polymer blended is 0.1% by mass or higher, a thickening mechanism by blending a high-temperature-stable polymer can be sufficiently expressed.

The cosmetic for use in a warmed state according to the present disclosure is preferably used under the temperature conditions at 30 to 70° C., and more preferably at 36 to 66° C. High actual feelings of effectiveness from the cosmetic is obtainable by using the cosmetic under the temperature conditions at 30 to 70° C. Moreover, since the cosmetic for use in a warmed state according to the present disclosure uses a temperature-responsive polymer and a high-temperature-stable polymer simultaneously, the cosmetic does not drip during use even by warming and separation can be suppressed.

A heat source for a heating part of a device using the cosmetic for use in a warmed state according to the present disclosure is not particularly limited, and a heater or a Peltier device is preferred.

Devices using a cosmetic for use in a warmed state are not particularly limited, and examples thereof are those comprising an atomizer such as a dispenser type atomizer equipped with a pump nozzle which can atomize the content of a container while keeping the inside pressure of the container at atmospheric pressure; an aerosol type atomizer in which a propellant is charged into the inside of a container; an ultrasonic atomizer in which mesh pores are oscillated at a high frequency; an ultrasonic atomizer in which a liquid column is formed from a liquid level; a multiple fluids mixing type atomizer in which another liquid is mixed with a cosmetic (the mixing may be performed either inside or outside the device); an electrostatic atomizer in which a cosmetic is turned into mist by an impact due to electrostatic pulse; and an air brush type atomizer in which a cosmetic is turned into mist from a needle tip by means of air flow. Further examples are devices equipped with a heating probe for use to the skin in a warmed state, devices equipped with a tank containing cosmetics for use in a warmed state, and devices for warming the tank that are used in an aesthetic salon, in the cosmetic medical field, or as home beauty devices.

The cosmetic for use in a warmed state according to the present disclosure is applicable to a beauty method such that when a cosmetic in a mist state is directly and/or indirectly applied to the skin using the above devices, specifically beauty devices used in an aesthetic salon, in the cosmetic medical field, or as home beauty devices, the cosmetic, of which temperature is controlled within a temperature range of 40 to 70° C., is used. The cosmetic for use in a warmed state according to the present disclosure is also applicable to a beauty method such that the cosmetic, of which temperature is controlled within a temperature range of 30 to 48° C. with the above devices, specifically a heating probe for use in a warmed state, is applied directly and/or indirectly applied to the skin.

Here, “indirectly” means that when the above devices or cosmetics for use in a warmed state are applied, the devices or cosmetics are applied to the skin through other cosmetics or cosmetic tools. An example of the case is such that a cosmetic for use in a warmed state, which was soaked into cotton, is applied onto the skin.

To the cosmetics and cosmetics for use in a warmed state according to the present disclosure, components ordinarily blended with cosmetics such as aqueous components, oily components, and powders may be blended. The cosmetics and cosmetics for use in a warmed state according to the present disclosure may be composed of an aqueous component as a main dispersion medium and may have an emulsification structure.

Examples of the aqueous components are water and water-soluble components. Examples of the water-soluble components are lower alcohols, moisturizers, and water-soluble polymers (such as native, semisynthetic, synthetic, and inorganic water-soluble polymers). The water-soluble polymers mean those not used for thickening.

Examples of the lower alcohols are ethanol, propanol, butanol, pentanol and hexanol.

Examples of the moisturizers are glycerin, diethylene glycol, butylene glycol, polyethylene glycol, hexylene glycol, xylitol, sorbitol, maltitol, chondroitin sulfate, hyaluronic acid, mucoitinsulfuric acid, caronic acid, atelocollagen, elastin, amino acid, nucleic acid, cholesteryl-12-hydroxystearate, sodium lactate, bile salts, dl-pyrrolidone carboxylate, short-chain soluble collagen, diglycerin (EO) PO adduct, Rosa roxburghii extract, Achillea millefolium extract, and Merrilot extract. EO is an abbreviation for ethylene oxide, and PO is an abbreviation for propylene oxide.

Examples of the native water-soluble polymers are vegetable-based water-soluble polymers such as gum arabic, tragacanth gum, galactan, guar gam, Locust bean gum, tamarind gum, carob gum, karaya gum, carageenan, pectin, agar, quince seed (marmelo), algecolloid (phaeophyta extract), starch (rice, maize, potato, wheat), and glycyrrhizic acid; microbe-based water-soluble polymers such as xanthan gum, dextran, succinoglycan, and pullulan; and animal-based water-soluble polymers such as collagen, casein, albumin, and gelatin.

Examples of the semisynthetic water-soluble polymers are starch-based water-soluble polymers such as carboxymethyl starch, and methylhydroxypropyl starch; cellulose-based water-soluble polymers such as methylcellulose, nitrocellulose, ethyl cellulose, methylhydroxypropyl cellulose, hydroxyethyl cellulose, sodium cellulose sulfate, hydroxypropyl cellulose, carboxymethyl cellulose (CMC), crystalline cellulose, and cellulose powder; alginic acid-based water-soluble polymers such as sodium alginate, and propyleneglycol alginate.

Examples of the synthetic water-soluble polymers are vinyl-based water-soluble polymers such as polyvinyl alcohol, polyvinyl methyl ether, polyvinylpyrrolidone, and carboxyvinyl polymer (Carbopol); polyoxyethylene-based water-soluble polymers such as polyethyleneglycol 20,000, polyethyleneglycol 4,000,000, and polyethyleneglycol 600,000; copolymer-based water-soluble polymers such as a polyoxyethylene-polyoxypropylene copolymer; acrylic water-soluble polymers such as sodium polyacrylate, polyethyl acrylate, and polyacrylamide; polyethyleneimine; and cation polymers.

Examples of the inorganic water-soluble polymers are bentonite, magnesium aluminum silicate (veegum), laponite, hectorite, and silicic anhydride.

As the powder components, both hydrophobic powder and hydrophilic powder may be used. Not only powders which are inherently hydrophobic or hydrophilic, but also powders having hydrophobized or hydrophilized surfaces may be used.

Examples of the powder components are talc, kaolin, mica, sericite, white mica, phlogopite, synthetic mica, lepidolite, biotite, lepidolite, vermiculite, magnesium carbonate, calcium carbonate, aluminum silicate, barium silicate, calcium silicate, magnesium silicate, strontium silicate, metal tungstate, magnesium, silica, zeolite, barium sulfate, calcined calcium sulfate (calcined gypsum), calcium phosphate, fluoroapatite, hydroxyapatite, ceramic powder, metal soaps (such as zinc myristate, calcium palmitate, and aluminum stearate), organic powders such as a polyamide resin powder (nylon powder), a polyethylene powder, a methyl polymethacrylate powder, a polystyrene powder, a styrene/acrylic acid copolymer resin powder, a benzoguanamine resin powder, a polytetrafluoroethylene powder, and a cellulose powder, silicone powders such as a trimethylsilsesquioxane powder, and inorganic powders such as boron nitride; inorganic white pigments such as titanium dioxide and zinc oxide; inorganic red pigments such as iron oxide (blood red) and iron titanate; inorganic brown pigments such as γ-iron oxide; inorganic yellow pigments such as yellow iron oxide and Chinese yellow; inorganic black pigments such as black iron oxide, carbon black, lower titanium dioxide; inorganic violet pigments such as mango violet, cobalt violet; inorganic green pigments such as chromium oxide, chromium hydroxide, and cobalt titanate; inorganic blue pigments such as ultramarine blue and iron blue; pearl pigments such as titanium dioxide-coated mica, titanium dioxide-coated bismuth oxychloride, titanium dioxide-coated talc, colored titanium dioxide-coated mica, bismuth oxychloride, and fish scale flake; and metal powder pigments such as aluminum powder, and copper powder.

Any method is applicable to a treatment for hydrophobizing the above powder components, as long as it can impart water repellency by any method, and ordinary surface treatment methods such as a gas-phase method, a liquid-phase method, an autoclave method, and mechanochemical method are applicable. Hydrophobizing agents are not particularly limited, and examples thereof are fatty acid dextrin-treated powder, trimethylsiloxy silicate-treated powder, fluorine-modified trimethylsiloxy silicate-treated powder, methylphenylsiloxy silicate-treated powder, fluorine-modified methylphenylsiloxy silicate-treated powder, powders treated with low-viscosity to high-viscosity oily polysiloxanes such as dimethylpolysiloxane, diphenylpolysiloxane, and methylphenylpolysiloxane, gummy polysiloxane-treated powder, methylhydrogenpolysiloxane-treated powder, fluorine-modified methylhydrogenpolysiloxane-treated powder, powders treated with organosilyl compounds or organosilyl compounds substituted with fluorine such as methyltrichlorosilane, methyltrialkoxysilane, hexamethyldisilane, dimethyldichlorosilane, dimethyldialkoxysilane, trimethylchlorosilane, and trimethylalkoxysilane, powders treated with modified organosilanes or modified organosilanes substituted with fluorine such as ethyltrichlorosilane, ethyltrialkoxysilane, propyltrichlorosilane, propyltrialkoxysilane, hexyltrichlorosilane, hexyltrialkoxysilane, long chain alkyltrichlorosilane, and long chain alkyltriethoxysilane, an amino-modified polysiloxane-treated powder, a fluorine-modified polysiloxane-treated powder, and a fluorinated alkyl phosphoric acid-treated powder.

Oily components blended with the cosmetic and the cosmetic for use in a warmed stated according to the present disclosure are not particularly limited as long as they can ordinarily be blended with cosmetics. Examples thereof are fats and oils, waxes, hydrocarbon oils, higher fatty acids, higher alcohols, synthetic ester oils, and silicone oils.

Examples of fats and oils are liquid fats and oils such as avocado oil, tea seed oil, evening primrose oil, turtle oil, macadamia nut oil, maize oil, mink oil, olive oil, rapeseed oil, egg-yolk oil, sesame oil, persic oil, wheat germ oil, Camellia sinensis leaf oil, castor oil, linseed oil, safflower oil, cotton seed oil, perilla oil, soybean oil, peanut oil, tea seed oil, kaya oil, rice bran oil, tung oil, Japanese tung oil, jojoba oil, germ oil, triglycerin, glycerin trioctanoate, and glycerin triisopalmitate; and solid fats and oils such as cacao butter, coconut oil, horse fat, hardened coconut oil, palm oil, beef tallow, mutton tallow, hardened beef tallow, palm kernel oil, lard, beef bone fat, Japan tallow kernel oil, hardened oil, neat's foot oil, Japan tallow, and hardened castor oil.

Examples of waxes are beeswax, candelilla wax, cotton wax, carnauba wax, bayberry wax, insects wax, spermaceti, montan wax, bran wax, lanoline, kapok wax, acetylated lanoline, liquid lanoline, sugarcane wax, lanolin fatty acid isopropyl ester, hexyl laurate, reduced lanoline, jojoba wax, hardened lanoline, shellac wax, POE lanoline alcohol ether, POE lanoline alcohol acetate, POE cholesterol ether, lanolin fatty acid polyethylene glycol, and POE hydrogenated lanoline alcohol ether. POE is an abbreviation for polyoxyethylene.

Examples of hydrocarbon oils are liquid paraffine, ozokerite, squalene, pristane, paraffine, ceresin, squalene, vaseline, and microcrystalline wax.

Examples of higher fatty acids are lauric acid, myristic acid, palmitic acid, stearic acid, behenic acid, oleic acid, 12-hydoxystearic acid, undecylenic acid, tall oil acid, isostearic acid, linolic acid, linoleic acid, eicosapentaenoic acid (EPA), and docosahexaenoic acid (DHA).

Examples of higher alcohols are linear alcohols such as lauryl alcohol, cetyl alcohol, stearyl alcohol, behenyl alcohol, myristyl alcohol, oleyl alcohol, and cetostearyl alcohol; and branched alcohols such as monostearylglycerin ether (batyl alcohol), 2-decyltetradecynol, lanolin alcohol, cholesterol, phytosterol, hexyl dodecanol, isostearyl alcohol, and octyl dodecanol.

Examples of synthetic ester oils are isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, 12-cholesteryl hydroxystearate, di-2-ethylhexyl acid ethylene glycol, dipentaerythritol fatty acid ester, N-alkylglycol monoisostearate, neopentyl glycol dicaprate, diisostearyl malate, glycerin di-2-hepthylundecanoate, trimethylolpropane tri-2-ethylhexylate, trimethylolpropane triisostearate, pentaerythritol tetra-2-ethylhexylate, glycerin tri-2-ethylhexylate, trimethylolpropane triisostearate, cetyl 2-hethylhexanoate, 2-ethylhexylpalmitate, glycerin trimyristate, glyceride tri-2-heptylundecanoate, castor oil fatty acid methyl ester, oleic acid oil, acetglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate, triethyl citrate, and crotamiton (C₁₃H₁₇NO).

Examples of the silicone oils are chain polysiloxanes such as dimethylpolysiloxane, methylphenylpolysiloxane, and methylhydrogenpolysiloxane; cyclic polysiloxanes such as decamethylpolysiloxane, dodecamethyl polysiloxane, and tetramethyltetrahydrogen polysiloxane; and silicone resin and silicone rubber forming a three-dimensional network structure.

As the emulsifiers, emulsifiers which may generally be blended with oil-in-water emulsion cosmetics may be blended. As the emulsifiers, those composed of one or more types having an HLB value of 8 or more are preferred. For example, one or more emulsifiers are blended which are selected from glycerin or polyglycerin fatty acid esters, propylene glycol fatty acid esters, POE sorbitan fatty acid esters, POE sorbit fatty acid esters, POE glycerin fatty acid esters, POE fatty acid esters, POE alkyl ethers, POE alkylphenyl ethers, POE.POP alkyl ethers, POE castor oil or hardened castor oil derivatives, POE honeybee wax/lanoline derivatives, alkanolamides, POE propylene glycol fatty acid esters, POE alkylamines, and POE fatty acid amides.

Blendable components other than the components described above are for example as follows: preservatives (such as ethylparaben, and butylparaben); anti-inflammatories (such as glycyrrhizic acid derivatives, glycyrrhezic acid derivatives, salicylic acid derivatives, Hinokitiol, zinc oxide, and allantoin); whitening agents (such as strawberry geranium extract, and arbutin); extracts (such as phellodendron bark, coptis root, lithospermi radix, paeoniae radix, swertiae herba, birch, sage, loquat, carrot, aloe, mallow, iris, grape, coix seed, loofah, lily, saffron, cnidium rhizome, Zingiber officinale, hypericum, Ononis spinosa, garlic, capsicum, citrus unshiu peel, Angelica acutiloba, and seaweeds); activators (such as royal jelly, photosensitizer, cholesterol derivatives); blood circulation accelerators (such as nonanoic acid vanillylamide, benzyl nicotinate, β-butoxyethylester nicotinate, capsaicin, zingerone, cantharides tincture, ichthammol, tannic acid, α-borneol, tocopherol nicotinate, inositol hexanicotinate, cyclandelate, cinnarizine, tolazoline, acetylcholine, Verapamil, cepharanthine, and γ-oryzanol); antiseborrheic agents (such as sulfur, and thianthol); anti-inflammatory agents (such as tranexamic acid, thiotaurine, and hypotaurine); and UV absorbers. However, blendable components are not limited to the above examples.

### EXAMPLES

The present disclosure is further specifically described based on the examples below. However, the present disclosure is not limited to the examples. All the blend amounts described herein are represented by % by mass unless otherwise specified.

In the present examples, the following compounds were used as (A) and (B).

(A): Hydrophobically Modified Polyether Urethane

A copolymer represented by the above formula (I) (in which R₁, R₂, and R₄ are ethylene groups, respectively, R₃ is a hexamethylene group, R₅ is 2-dodecyldodecyl group, h is 1, m is 2, k is 120, and n is 20) ((PEG-240/decyltetradeces-20/HDI) copolymer: “ADEKANOL GT-700” produced by ADEKA Corporation) was used.

(B): Cellulose Nanofibers

A fine fibrous cellulose having a maximum fiber diameter of 1,000 nm or less (“REOCRYSTA C-2SP” produced by DSK Co., Ltd) was used. REOCRYSTA C-2SP is a product containing 2% by mass of fine fibrous cellulose and 1% by mass of phenoxyethanol (preservative) in 97% by mass of water. The mass % values described in the present specification and tables show values of the fine fibrous cellulose only, not including the values of water and the preservative contained in the product.

### Examples for Cosmetic

Cosmetics were prepared by blending components in accordance with the prescription shown in Table 4 such that relative to the total amount of each cosmetic, the blend amounts of (A) alone, (A) and (B), and (B) alone are 2% by mass, the blend amounts of (A) alone, (A) and (B), and (B) alone are 1.75% by mass, and the blend amounts of (A) alone, (A) and (B), and (B) alone are 0.75% by mass, and in each of the concentrations, the blend ratios of (A) to (B) were as described in Tables 1 to 3 below. On the obtained samples, the following coating test and dispenser test were performed. The evaluation results are summarized in Tables 1 to 3.

(Coating Test)

On a glass petri dish with φ50 mm, 10 g of each sample prepared under each of the blending conditions was dropped and left in a flat surface state at 50° C. for 24 hours, and thereafter the state of the sample was evaluated in accordance with the criteria below:

A: Sample being peeled off as a solid from the petri dish

C: Sample not being peeled off from the petri dish due to stickiness

(Dispenser Test)

The continuous dispensing of each sample prepared under each of the blending conditions was evaluated using a dispenser with 0.7 mL dispensing in accordance with the criteria below:

A: Continuously dispensed without air inclusion

B: Continuously dispensed by press operations up to 5 times

C: Discontinuously dispensed

TABLE 1

| Blend amount (A) + | | | | | | | |
|---|---|---|---|---|---|---|---|
| (B) 2% by mass | (A) | (A) > (B) | | (A) = (B) | (A) < (B) | | (B) |
| Blend ratio (A):(B) | 100:0 | 90:10 | 75:25 | 50:50 | 25:75 | 10:90 | 0:100 |
| Coating test | C | A | A | A | A | A | A |
| Dispenser test | A | B | C | C | C | C | C |

TABLE 2

| Blend amount (A) + | | | | | | | |
|---|---|---|---|---|---|---|---|
| (B) 1.75% by mass | (A) | (A) > (B) | | (A) = (B) | (A) < (B) | | (B) |
| Blend ratio (A):(B) | 100:0 | 90:10 | 75:25 | 50:50 | 25:75 | 10:90 | 0:100 |
| Coating test | C | A | A | A | A | A | A |
| Dispenser test | A | A | A | B | C | C | C |

TABLE 3

| Blend amount (A) + | | | | | | | |
|---|---|---|---|---|---|---|---|
| (B) 0.75% by mass | (A) | (A) > (B) | | (A) = (B) | (A) < (B) | | (B) |
| Blend ratio (A):(B) | 100:0 | 90:10 | 75:25 | 50:50 | 25:75 | 10:90 | 0:100 |
| Coating test | C | A | A | A | A | A | A |
| Dispenser test | A | A | A | A | A | B | C |

TABLE 4

| Components | |
|---|---|
| Ion exchange water | remainder |
| Ethanol | 7 |
| (A) (PEG-240/decyltetradeces-20/HDI) copolymer | See Tables 1 to |
| (B) Microfibrous cellulose | 3 and Table 5 |
| Citric acid | 0.01 |
| Sodium citrate | 0.09 |
| Blend amount (% by mass) | 100 |

The coating test shows that greasiness is suppressed when (B) is blended. This result was not obtained with the sample in which (A) alone was blended in an amount of 2% by mass shown in Table 1. The dispenser test shows that when the blend ratio of (A) is higher, the sample cosmetic is dispensable even when higher amounts of (A) and (B) are blended. In contrast, when the blend ratio of (B) is high, dispensing is difficult. This means that a cosmetic approximates a discontinuous body, namely an elastic body due to the blending of (B). Thus, the above shows that a preparation without greasiness, which is dispensable from a dispenser, can be obtained by combining (A) and (B), even when a high amount of (A) is blended.

Namely, the appropriate values of the combination are:

when the blend ratio is such that (A)<(B), the amount of (A)+(B) blended relative to the total cosmetic is 0.75% by mass or lower;

when the blend ratio is such that (A)=(B), the amount of (A)+(B) blended relative to the total cosmetic is 1.75% by mass or lower; and

when the blend ratio is such that (A)>(B), the amount of (A)+(B) blended relative to the total cosmetic is 2% by mass or lower.

### Examples of Cosmetics for Use in Warmed State

Cosmetics were prepared by blending components in accordance with the prescription shown in Table 4 such that relative to the total amount of each cosmetic, the blend amounts of (A) alone, (A) and (B), and (B) alone are 1% by mass, respectively, and the blend ratios of (A) to (B) were as described in Table 5 below. Each of the prepared samples was subjected to dynamic viscoelasticity measurement using a controlled stress rheometer MCR301 produced by Anton Paar. Under the measurement conditions of a cone plate with φ25 mm and temperatures at 30° C. and 60° C., a storage elastic modulus G′ and a loss elastic modulus G″ when a distortion was increased from 0.01 to 5,000 were measured.

In general, solid properties are regarded as predominant physical properties when G′>G″ (when G″/G′≤1), and liquid properties are regarded as predominant physical properties when G″>G′ (when G″/G′≥1). Since properties when a distortion is small particularly contribute to the stability against dripping and separation, the evaluation was performed based on physical properties when a distortion value was 1.

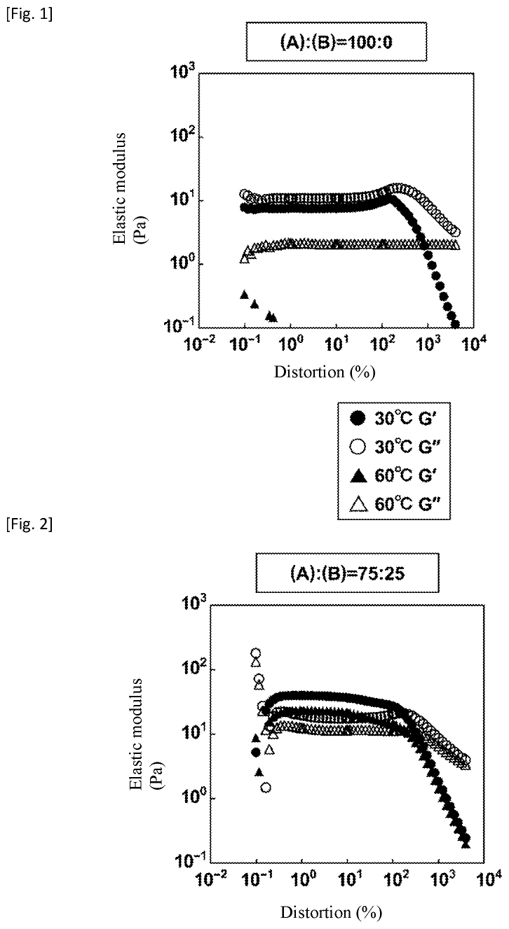

The results are summarized in Table 5 and FIGS. 1 to 5. As described under the graph of FIG. 1, • means G′ at 30° C., ∘ means G″ at 30° C., ▴ means G′ at 60° C., and Δ means G″ at 60° C.

TABLE 5

| Blend amount | | | | | | | | | | |
|---|---|---|---|---|---|---|---|---|---|---|
| (A) + (B) | | | | | | | | | | |
| 1% by mass | | (A) | | | (A) > (B) | | | (A) = (B) | | |
| Blend ratio (A):(B) | | 100:0 | | | 75:25 | | | 50:50 | | |
| Elastic modulus | | G′ | G″ | G″/G′ | G′ | G″ | G″/G′ | G′ | G″ | G″/G′ |
| Distortion | 30° C. | 7.66 | 10.90 | 1.42391 | 39.75 | 19.65 | 0.49434 | 136.50 | 26.95 | 0.19744 |
| 1 | 60° C. | 0.07 | 2.15 | 32.2072 | 23.30 | 12.55 | 0.53863 | 151.00 | 31.85 | 0.21093 |
| | | | Blend amount | | | | | | | |
| | | | (A) + (B) | | | | | | | |
| | | | 1% by mass | | (A) < (B) | | | (B) | | |
| | | | Blend ratio (A):(B) | | 25:75 | | | 0:100 | | |
| | | | Elastic modulus | | G′ | G″ | G″/G′ | G′ | G″ | G″/G′ |
| | | | Distortion | 30° C. | 293.00 | 36.25 | 0.12372 | 538.30 | 45.80 | 0.08505 |
| | | | 1 | 60° C. | 338.00 | 46.25 | 0.13683 | 659.50 | 63.95 | 0.09697 |

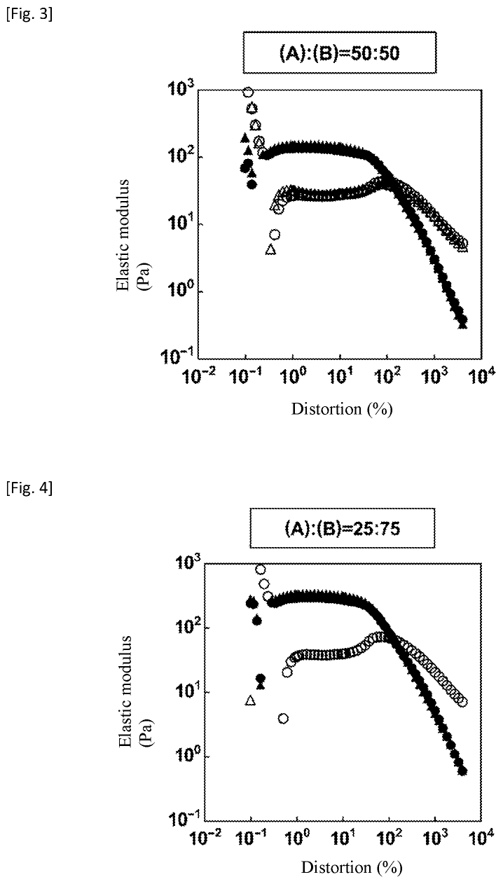

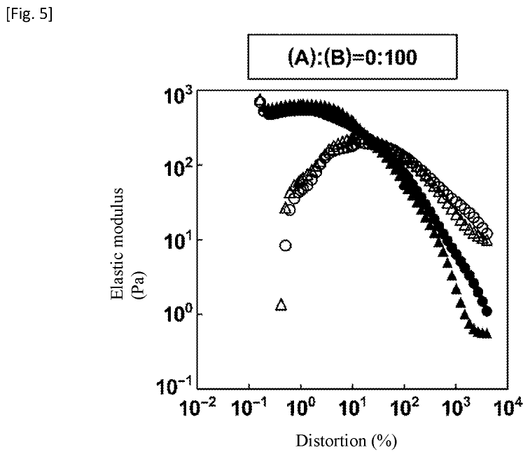

When only (A) (=temperature-responsive polymer) was blended, an elastic modulus was decreased and the loss elastic modulus G″ became remarkably predominant when the temperature was increased from 30° C. to 60° C. (cf. FIG. 1). In contrast, when only (B) (=cellulose nanofiber) was blended, an elastic modulus did not change before and after warming, and the storage elastic modulus G′ was high (cf. FIG. 5). When (B)>(A), the physical properties of (B) were predominant and the change of an elastic modulus was small before and after warming while maintaining a high elastic modulus (cf. FIG. 4). When (A)>(B), an elastic modulus was decreased during warming, and the storage elastic modulus G′ was also high at high temperatures and solid properties were maintained (cf. FIG. 2). When (A)=(B), an elastic modulus did not change before and after warming (cf. FIG. 3). The above results show that in order to improve the temperature stability while controlling the afterfeel accompanied by viscosity change during warming, the condition (A)>(B) in which an elastic modulus is decreased during warming and simultaneously G′>G″ is satisfied at low distortion values is preferred.

(Formulation Examples of Water-Dispersed Cosmetics)

In accordance with the formulations shown in Table 6, the components other than ion exchange water were dissolved and dispersed in ion exchange water to prepare formulation examples 1 to 12 of water-dispersed cosmetics.

TABLE 6

| Components | No. 1 | No. 2 | No. 3 | No. 4 | No. 5 | No. 6 | No. 7 |
|---|---|---|---|---|---|---|---|
| Ion exchange water | remainder | remainder | remainder | remainder | remainder | remainder | remainder |
| Ethanol | 7 | 7 | 7 | 7 | 7 | 7 | 7 |
| Glycerin | 5 | 5 | 5 | 5 | 5 | 5 | 5 |
| Dipropyleneglycol | 8 | 8 | 8 | 8 | 8 | 8 | 8 |
| (A) (PEG-240/ | 0.2 | 0.125 | 0.05 | 0.9 | 0.375 | 0.025 | 1.35 |
| decyltetradeces- | | | | | | | |
| 20/HDl) copolymer | | | | | | | |
| (B) Microfibrous | 0.05 | 0.125 | 0.2 | 0.1 | 0.125 | 0.225 | 0.15 |
| cellulose | | | | | | | |
| PPG-13 | 0.2 | 0.2 | 0.2 | 0.2 | 0.2 | 0.2 | 0.2 |
| decyltetradeces-24 | | | | | | | |
| Citric acid | 0.01 | 0.01 | 0.01 | 0.01 | 0.01 | 0.01 | 0.01 |
| Sodium citrate | 0.09 | 0.09 | 0.09 | 0.09 | 0.09 | 0.09 | 0.09 |
| Phenoxyethanol | 0.2 | 0.2 | 0.2 | 0.2 | 0.2 | 0.2 | 0.2 |
| Perfume | 0.01 | 0.01 | 0.01 | 0.01 | 0.01 | 0.01 | 0.01 |
| Blend amount | 100 | 100 | 100 | 100 | 1 00 | 100 | 100 |
| (% by mass) | | | | | | | |
| | | Components | No. 8 | No. 9 | No. 10 | No. 11 | No. 12 |
| | | Ion exchange water | remainder | remainder | remainder | remainder | remainder |
| | | Ethanol | 7 | 7 | 7 | 7 | 7 |
| | | Glycerin | 5 | 5 | 5 | 5 | 5 |
| | | Dipropyleneglycol | 8 | 8 | 8 | 8 | 8 |
| | | (A) (PEG-240/ | 0.75 | 0.25 | 1.125 | 0.75 | 0.15 |
| | | decyltetradeces- | | | | | |
| | | 20/HDl) copolymer | | | | | |
| | | (B) Microfibrous | 0.25 | 0.25 | 0.375 | 0.75 | 0.6 |
| | | cellulose | | | | | |
| | | PPG-13 | 0.2 | 0.2 | 0.2 | 0.2 | 0.2 |
| | | decyltetradeces-24 | | | | | |
| | | Citric acid | 0.01 | 0.01 | 0.01 | 0.01 | 0.01 |
| | | Sodium citrate | 0.09 | 0.09 | 0.09 | 0.09 | 0.09 |
| | | Phenoxyethanol | 0.2 | 0.2 | 0.2 | 0.2 | 0.2 |
| | | Perfume | 0.01 | 0.01 | 0.01 | 0.01 | 0.01 |
| | | Blend amount | 100 | 100 | 100 | 100 | 100 |
| | | (% by mass) | | | | | |

(Formulation Examples of Emulsion Cosmetics)

In accordance with the formulations shown in Table 7, oily components and aqueous components were individually dissolved and mixed, thereafter the oily components were mixed with the aqueous components to effectuate emulsification, and thereby the formulation examples 1 to 5 of the emulsion cosmetics were prepared.

TABLE 7

| Components | No. 1 | No. 2 | No. 3 | No. 4 | No. 5 |
|---|---|---|---|---|---|
| Ion exchange water | remainder | remainder | remainder | remainder | remainder |
| Glycerin | 4 | 5 | 5 | 4 | 5 |
| Dipropylene glycol | 9 | 7 | 7 | 5 | 2 |
| BG | 4 | — | — | 2 | 6 |
| PEG-6 | 0.5 | — | — | — | 0.5 |
| PEG-32 | 0.5 | — | — | — | 0.5 |
| PEG/PPG-14/7 dimethylether | 1 | — | — | 0.1 | — |
| Isostearic acid PEG-60 glyceryl | — | — | — | 1.3 | — |
| Stearic acid PEG-5 glyceryl | — | — | — | 0.7 | — |
| PEG-60 hydrogenated castor oil | — | — | — | — | 0.5 |
| (A) (PEG-240/decyltetradeces- | 0.9 | 0.2 | 0.125 | 0.2 | 0.04 |
| 20/HDI) copolymer | | | | | |
| (B) Microfibrous cellulose | 0.1 | 0.05 | 0.125 | 0.2 | 0.16 |
| Sodium methyl stearoyl taurate | — | 0.9 | 0.9 | — | — |
| Behenyl alcohol | 0.08 | 4.8 | 4.8 | 0.6 | 0.5 |
| Cetearyl alcohol | — | 1.4 | 1.4 | — | — |
| Batyl alcohol | 0.03 | — | — | 0.15 | 0.1 |
| Pentaerythrityl tetraethylhexanoate | 0.2 | 5 | 5 | 2 | 2 |
| Vaseline | — | — | — | 1 | 4 |
| Squalane | — | — | — | 2 | 4 |
| Dimethicone | — | — | — | 4 | 1 |
| Cetyl ethylhexanoate | — | 5 | 5 | — | — |
| Ethylhexyl methoxycinnamate | — | — | 5 | — | — |
| t-butylmethoxydibenzoylmethane | — | — | 2 | — | — |
| Citric acid | 0.01 | 0.01 | 0.01 | 0.01 | 0.01 |
| Sodium citrate | 0.09 | 0.09 | 0.09 | 0.09 | 0.09 |
| Phenoxyethanol | 0.2 | 0.2 | 0.2 | 0.3 | 0..3 |
| Perfume | 0.01 | 0.01 | 0.01 | 0.01 | 0.01 |
| Blend amount (% by mass) | 100 | 100 | 100 | 100 | 100 |

(Formulation Examples of Powder Blended Cosmetics)

In accordance with the formulations shown in Table 8, oily components and aqueous components were individually dissolved and mixed, thereafter a powder conformable to oil and a powder conformable to water are dispersed in oily components and aqueous components, respectively, and the oily components were mixed with the aqueous components to effectuate emulsification, and thereby the formulation examples 1 to 5 of the powder blended cosmetics were prepared.

TABLE 8

| Components | No. 1 | No. 2 | No. 3 | No. 4 | No. 5 |
|---|---|---|---|---|---|
| Ion exchange water | remainder | remainder | remainder | remainder | remainder |
| Ethanol | 20 | 10 | 20 | 6 | 6 |
| Glycerin | 5 | 1 | 5 | 1 | 1 |
| Dipropylene glycol | — | 4 | 5 | — | — |
| BG | — | — | — | 4 | 4 |
| PEG-12 dimethicone | — | — | — | 0.4 | 0.4 |
| PEG/PPG-14/7 dimethyl ether | 1 | 1 | 1 | — | — |
| Isostearic acid | — | — | — | 0.2 | 0.2 |
| PEG-60 hydrogenated castor oil | — | — | — | 1 | 1 |
| Sorbitan sesquiisostearate | — | — | — | 0.5 | 0.5 |
| PPG-13 decyltetradeces-24 | 0.02 | 0.02 | 0.02 | — | — |
| (A) (PEG-240/decyltetradeces- | 0.125 | 0.025 | 0.125 | 0.375 | 0.225 |
| 20/HDI) copolymer | | | | | |
| (B) Microfibrous cellulose | 0.125 | 0.225 | 0.375 | 0.125 | 0.025 |
| Hydroxypropyl methylcellulose | — | — | — | 0.04 | 0.04 |
| stearoxy ether | | | | | |
| Kaolin | 1 | 1 | — | — | — |
| Bentonite | 0.5 | — | — | — | — |
| Silica | 1 | 5 | — | — | — |
| Titanium oxide | — | 7 | — | — | 10 |
| Zinc oxide | — | — | — | 9 | — |
| Iron oxide | — | 2 | 30 | — | 2 |
| Dimethicone | — | — | — | 4 | 15 |
| Diphenylsiloxy phenyl trimethicone | — | — | — | — | 5 |
| Diisopropyl sebacate | — | — | — | 8 | — |
| Sucrose tetrastearate triacetate | — | — | — | 2 | — |
| Ethylhexyl methoxycinnamate | — | — | — | 1 | 5 |
| Bis-ethylhexyloxyphenol | — | — | — | 2 | — |
| methoxyphenyl triazine | | | | | |
| Diethylamino hydroxybenzoyl | — | — | — | 2 | — |
| hexyl benzoate | | | | | |
| Ethylhexyl triazone | — | — | — | 1 | — |
| Sodium chloride | 0.3 | 0.3 | 0.3 | — | — |
| Sodium metaphosphate | — | 0.2 | — | — | — |
| Phenoxyethanol | 0.2 | 0.2 | — | 0.5 | 0.5 |
| Perfume | 0.01 | 0.01 | — | 0.1 | 0.1 |
| Blend amount % by mass) | 100 | 100 | 100 | 100 | 100 |

1. A cosmetic comprising
(A) a hydrophobically modified polyether urethane,
(B) cellulose nanofibers, and
(C) water,
wherein,
when the blend ratio of the (A) hydrophobically modified polyether urethane to the (B) cellulose nanofibers is such that (A)<(B), the amount of (A)+(B) blended relative to the total cosmetic is 0.75% by mass or lower,
when the blend ratio of (A) to (B) is such that (A)=(B), the amount of (A)+(B) blended relative to the total cosmetic is 1.75% by mass or lower, and
when the blend ratio of (A) to (B) is such that (A)>(B), the amount of (A)+(B) blended relative to the total cosmetic is 2% by mass or lower.

2. The cosmetic according to claim 1, wherein the (A) hydrophobically modified polyether urethane is a (PEG-240/decyltetradeces-20/HDI) copolymer.

3. The cosmetic according to claim 1, wherein the (B) cellulose nanofibers are microfibrous cellulose having a maximum fiber diameter of 1,000 nm or less.

4. The cosmetic according to claim 1, being contained in a dispenser container.

5. A cosmetic for use in a warmed state that is used in a device having a heating part, said cosmetic comprising
a temperature-responsive polymer having a structure changing at a temperature of 30° C. or higher,
a high-temperature-stable polymer having a structure not changing at a temperature of 70° C. or lower, and
water.

6. The cosmetic for use in a warmed state according to claim 5, wherein the temperature-responsive polymer is (A) a hydrophobically modified polyether urethane and the high-temperature-stable polymer is (B) cellulose nanofibers.

7. The cosmetic for use in a warmed state according to claim 6, wherein the amount of the temperature-responsive polymer blended is higher than the amount of the high-temperature-stable polymer blended, and the amount of the high-temperature-stable polymer blended relative to the total cosmetic is 0.1% by mass or higher.

8. The cosmetic for use in a warmed state according to claim 6, wherein the (A) hydrophobically modified polyether urethane is a (PEG-240/decyltetradeces-/HDI) copolymer.

9. The cosmetic for use in a warmed state according to claim 6, wherein the (B) cellulose nanofibers consist of microfibrous cellulose having a maximum fiber diameter of 1,000 nm or less.

10. The cosmetic for use in a warmed state according to claim 5, being used under temperature conditions at 30 to 70° C.

11. The cosmetic for use in a warmed state according to claim 5, wherein the heating part has a heat source which is a heater or a Peltier device.

12. The cosmetic for use in a warmed state according to claim 5, wherein the device is equipped with an atomizer.

13. The cosmetic for use in a warmed state according to claim 5, wherein the device is equipped with a probe.

14. The cosmetic for use in a warmed state according to claim 5, wherein the device is equipped with a tank for containing the cosmetic for use in a warmed state.

15. A beauty method wherein the temperature of the cosmetic for use in a warmed state according to claim 5 is controlled within a temperature range of 40 to 70° C. in the heating part and the cosmetic is directly and/or indirectly applied in a mist state onto the skin.

16. A beauty method wherein the temperature of the cosmetic for use in a warmed state according to to claim 5 is controlled within a temperature range of 30 to 48° C. in the heating part and the cosmetic is directly and/or indirectly applied in a mist state onto the skin.
